(19) United States
(12) Patent Application Publication
(10) Pub. No.: US 2026/0144597 A1
(43) Pub. Date: May 28, 2026

(54) TISSUE PROPERTY LOCALIZATION FOR THERAPY DELIVERY

(71) Applicant: Medtronic, Inc., Minneapolis, MN (US)

(72) Inventor: Elliot C. Schmidt, Minneapolis, MN (US)

(21) Appl. No.: 19/150,075

(22) PCT Filed: Jan. 11, 2024
(86) PCT No.: PCT/IB2024/050283

§ 371 Date: Jul. 22, 2025

Related U.S. Application Data
(60) Provisional application No. 63/481,791, filed on Jan. 26, 2023.

(51) Int. Cl. A61B 34/20; A61N 1/362; G16H 20/40

(52) U.S. Cl. CPC A61B 34/20; G16H 20/40; A61B 2034/2065; A61N 1/362

(57) ABSTRACT

An example system includes memory configured to store a representation of patient tissue and processing circuitry. The processing circuitry is configured to determine, via a mechanical response, at least one property of patient tissue at a location relative to a reference position. The processing circuitry is configured to localize, using the reference position, the patient tissue relative to at least one of a medical instrument or a medical device. The processing circuitry is configured to output, for display, a representation of at least one of the medical instrument or the medical device and a representation of the patient tissue, the representation of the patient tissue comprising a representation of the at least one property.

This application claims the benefit of U.S. Provisional Patent Application Ser. No. 63/481,791, filed Jan. 26, 2023, the entire content of which is incorporated herein by reference.

### FIELD

The present disclosure relates to medical imaging, and in particular to medical imaging to guide the navigation of medical instrument(s) and/or device(s).

### BACKGROUND

An imaging system may be used to image various portions of a subject. The subject may include a patient, such as a human patient. The portions of the patient selected to be imaged may be internal portions that are covered by skin or other tissue. An imaging system may include, for example, an imaging device, such as an ultrasound imaging transducer, and may produce an image of patient tissue in a body of the patient, which may be displayed on a display, for example, in an operating room. The imaging system may also produce an image of a medical instrument or medical device, such as an implantable medical device (IMD), relative to the patient tissue, such as during an implant procedure. The location of the patient tissue and the medical instrument or medical device may be defined or established relative to each other (e.g., a location of a heart wall relative to a catheter).

The imaging system may display the imaging data on a display which may be viewed by a clinician, such as a clinician implanting an IMD into the patient. The clinician may use the displayed imaging data to guide navigation of the medical instrument or IMD during the implantation procedure.

### SUMMARY

This disclosure relates to an imaging system that may determine one or more properties of tissue of a patient and output indications of the one or more properties of the tissue such that the location and a representation of one or more properties of the tissue may be displayed on a display of an imaging system. For example, a system may determine a stiffness of the tissue and/or a fat content of the tissue. The stiffness and/or fat content of the tissue may be represented on the display, for example, using different colors.

For example, when implanting an IMD, such as a leadless pacemaker, in cardiac tissue of a patient, it may be desirable to implant the IMD (or electrodes associated therewith) at an appropriate location within or on the cardiac tissue such that efficacious therapy may be delivered by the IMD. Some tissue may less appropriate than other tissue for delivery of therapy. For example, scar tissue and fatty tissue may be less appropriate for delivery of electrical therapy, and thereby make a less appropriate an implantation site, than normal, healthy tissue (e.g., muscle tissue), as scar tissue and fatty tissue may be poorer electrical conductors than normal, healthy tissue.

Scar tissue may be generally stiffer than surrounding healthy tissue. Fatty tissue may be generally less stiff than surrounding healthy tissue. It may be desirable to determine a stiffness (which may be an indication of scar tissue and/or a fat content of tissue) and display indications of a stiffness, scarring, and/or fat content of tissue on a display during implantation of an IMD, such that a clinician or robotic device may guide a delivery catheter or other delivery instrument to more appropriate tissue for implantation and avoid highly scarred tissue and highly fatty tissue. For example, a system may output for display areas of tissue that are stiffer, more scarred, and/or have a higher fat content in one or more colors that may differ from areas that are less stiff, less scarred, or have a lower fat content. Such displayed content may facilitate a clinician to implant an IMD at a location within a patient that may provide more efficacious electrical therapy than other potential implantation locations.

A medical instrument, such as a delivery catheter, and/or a medical device, such as an IMD or lead, may be tracked relative to anatomy (e.g., the patient tissue) of the patient. A representation of the medical instrument and/or device may be superimposed or otherwise included in the displayed anatomy.

In one example, a system includes: memory configured to store a representation of patient tissue; and processing circuitry communicatively coupled to the memory, the processing circuitry being configured to: determine, via a mechanical response, at least one property of the patient tissue at a location relative to a reference position; localize, using the reference position, the patient tissue relative to at least one of a medical instrument or a medical device; and output, for display, a representation of at least one of the medical instrument or the medical device and a representation of the patient tissue, the representation of the patient tissue comprising a representation of the at least one property.

In one example, a method includes: determining, via a mechanical response and by an imaging system, at least one property of patient tissue at a location relative to a reference position; localizing, by the imaging system, using the reference position, the patient tissue relative to at least one of a medical instrument or a medical device; and outputting, by the imaging system, for display a representation of at least one of the medical instrument or the medical device and a representation of the patient tissue, the representation of the patient tissue comprising a representation of the at least one property.

In one example, a non-transitory computer-readable storage medium stores instructions, which, when executed, cause processing circuitry to: determine, via a mechanical response, at least one property of patient tissue relative to a reference coordinate; localize, using the reference coordinate, the patient tissue relative to at least one of a plane of a medical instrument or a medical device; and output, for display, a representation of at least one of the medical instrument or the medical device and a representation of the patient tissue, the representation of the patient tissue comprising a representation of the at least one property.

Further areas of applicability will become apparent from the description provided herein. The description and specific examples in this summary are intended for purposes of illustration only and are not intended to limit the scope of the present disclosure.

### BRIED DESCRIPTION OF DRAWINGS

FIG. 1 is an environmental view of a patient with an imaging and navigation system.

FIG. 2 is a schematic view of an example navigation processing system according to one or more aspects of this disclosure.

FIG. 3 is a block diagram illustrating an example mechanical response detection device according to one or more aspects of this disclosure.

FIG. 4 is a conceptual diagram illustrating a display of a medical instrument, IMD, and patient tissue including a representation of at least one property of the patient tissue according to one or more aspects of this disclosure.

FIG. 5 is a flow diagram illustrating example tissue property identification techniques in accordance with one or more aspects of this disclosure.

### DETAILED DESCRIPTION

Several different therapies may rely on avoiding or targeting bodily tissues having specific properties. For example, efficacious ventricle-from-atrium stimulation, e.g., pacing, therapy may be dependent on a pacemaker electrode avoiding fatty tissue between the right atrium and the left ventricle, efficaciousness of therapy provided by an extravascular implantable cardiac defibrillator may be dependent on avoiding or working through scar tissue adhesions, and the efficaciousness of therapy delivered by pacing leads may be dependent on avoiding myocardial scarring. Traditionally, fluoroscopic images, which provide no information relating to these types of soft-tissue properties, guide a clinician during the implantation of IMDs or leads for such therapies.

Traditional imaging systems used for navigation during medical procedures typically do not provide information relating to particular properties of patient tissue that may be useful for a clinician during implantation of an IMD or surgical treatment of the patient. For example, patient tissue that is more fatty or more scarred than other patient tissue may be less conductive to electrical signals, such as electrical stimulation therapy signals. Traditional imaging systems do not display such properties of patient tissue. As scar tissue and fatty tissue may be less conductive than healthy, normal tissue, it may be desirable to avoid such tissue as an implantation site or attachment site for electrode(s) of an IMD (or an electrode of a lead implanted in the patient for an external medical device). According to the techniques of this disclosure, an imaging system may determine at least one property of patient tissue, such as stiffness or fat content, and may output for display a representation of the patient tissue including information indicative of the at least one property, thereby enhancing the ability of a clinician (or a robot) to guide an IMD or other device or instrument to a more appropriate locations, such as to healthy tissue.

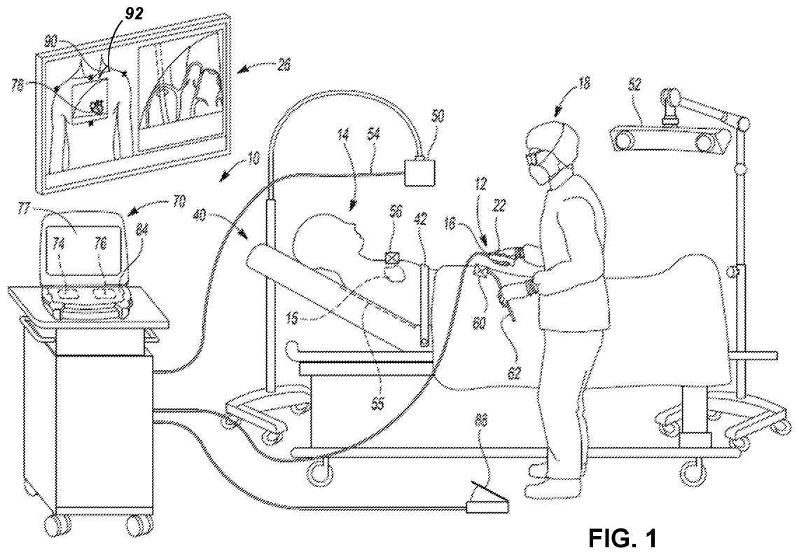

FIG. 1 is a diagram illustrating an overview of a navigation system 10 that may be used for various medical procedures. Navigation system 10 may be used to track the location of an item, such as an IMD and/or a medical instrument, relative to anatomy of a subject, such as a patient 14. It should be noted that navigation system 10 may be used to navigate any type of medical instrument, IMD, or delivery system, including: catheters, stylets, imaging devices, leads for cardiac rhythm management devices such as pacemakers, leadless pacemakers and delivery systems therefor (e.g., ventricle-from-atrium leadless pacemakers and delivery systems therefor), guide wires, arthroscopic systems, ablation instruments, stents, orthopedic implants, spinal implants, deep brain stimulation (DBS) probes, mechanical parts, etc. Non-human or non-surgical procedures may also use navigation system 10 to track a non-surgical or non-human intervention of an IMD and/or medical instrument. Moreover, the medical instruments may be used to navigate to and/or map any region of the body. Navigation system 10 and the various tracked items may be used in any appropriate procedure, such as one that is generally minimally invasive or an open procedure.

Navigation system 10 may interface with or integrally include an imaging system 12 that is used to acquire pre-operative, intra-operative, post-operative, and/or real-time image data of patient 14. For example, imaging system 12 may include an ultrasound imaging system (as discussed further herein) that has a tracking device 22 attached thereto. While this disclosure primarily discusses imaging system 12 as including an ultrasound imaging system, imaging system 12 may include other types of imaging systems such as a thermoacoustic imaging system, a fluoroscopic imaging system, or any other suitable imaging system.

Tracking device 22 may be tracked with the tracking system to determine a pose of imaging system 12. The pose may include an orientation (e.g., three or more degrees of orientation (e.g., yaw, pitch, and roll) and/or a position (e.g., three degrees of freedom in physical space (e.g., x-axis, y-axis, and z-axis). The tracking system may further determine appropriate pose information regarding the tracking device 22. The pose of imaging system 12 may then be determined based on the tracked pose of the tracking device 22. Imaging system 12 may be used to generate image data to provide images for viewing with a selected display device 26.

It will be understood any appropriate subject may be imaged and any appropriate procedure may be performed relative to the subject. Navigation system 10 may be used to track various tracking devices, as discussed herein, to determine locations of IMDs, medical instruments, imaging system 12, or the like, with respect to anatomy of patient 14. The tracked poses of patient 14 may be used to determine or select images for display to be used with the navigation system 10.

In the example of FIG. 1, imaging system 12 includes an ultrasound (US) imaging system with an US housing 16 that is held by a user 18 (e.g., a clinician) while collecting image data of patient 14. It will be understood, however, that US housing 16 may also be held by a stand, a robotic system, or the like, while collecting image data. While shown as an external US housing, in some examples, US housing 16 may be configured to be at least partially inserted into patient 14. For example, imaging system 12 may be an intravascular ultrasound (IVUS) system or other US system configured to be at least partially inserted into patient 14. Associated with, such as attached directly to or molded into, the US housing 16, or an US transducer housed within US housing 16, is at least one tracking device 22. Tracking device 22 may be any appropriate tracking device such as an electromagnetic tracking device and/or an optical tracking device. In some examples, tracking device 22 may include both an electromagnetic tracking device and an optical tracking device. It should be understood that various other tracking devices may be associated with the US housing 16, as discussed herein, including acoustic, ultrasound, radar, and other tracking devices.

Patient 14 may be fixed in a pose relative to a selected object, such as onto an operating table 40, but is not required to be fixed to table 40. Table 40 may include a plurality of straps 42. Straps 42 may be secured around patient 14 to generally fix patient 14 relative to table 40. Various other apparatuses may be used to position patient 14 in a static position on operating table 40.

Navigation system 10 includes at least one tracking system. The tracking system may include at least one localizer. In one example, the tracking system may include an electromagnetic (EM) localizer 50. The tracking system may be used to track IMDs, medical instruments, imaging systems, or the like relative to patient 14 or within a navigation space. Navigation system 10 may use image data from imaging system 12 and information from the tracking system to illustrate locations of the tracked devices, as discussed herein. The tracking system may also include a plurality of types of tracking systems including an optical localizer 52, in addition to, or in place of, EM localizer 50. When EM localizer 50 is used, EM localizer 50 may communicate with other devices of navigation system 10 through a localizer communication 54 that may be wired or wireless. In some examples, a pad or flat EM localizer 55 may be used in addition to or in place of EM localizer 50, which may be placed on table 40.

Optical localizer 52, EM localizer 50, and/or EM localizer 55 may be used together to track multiple devices or used together to redundantly track the same device. Various tracking devices, including those discussed further herein, may be tracked and tracking information generated by the tracking system may be used by navigation system 10 to allow for an output system to output, such as display device 26 to display, a position of one or more devices. Tracking devices may include a patient or reference tracking device 56 to track patient 14, an instrument tracking device 60 to track an instrument 62, and/or other appropriate tracking devices for tracking medical instruments, IMDs, patient anatomy, or portions thereof. The tracking devices allow selected portions of the operating theater to be tracked relative to one another with the appropriate tracking system, including optical localizer 52, EM localizer 50, and/or EM localizer 55. Reference tracking device 56 may alternatively be positioned on a medical instrument and be positioned within patient 14, such as within a heart 15 of patient 14. In some examples, navigation system 10 may determine a reference position, such as a reference coordinate, for example, based on a location of a device, such as reference tracking device 56 or EM localizer 50, which may be used to localize medical instruments, IMDs, patient tissue, or the like.

It will be understood that any of the tracking devices 22, 56, 60 may be optical or EM tracking devices, or both, depending upon the tracking localizer used to track the respective tracking devices. It will be further understood that any appropriate tracking system may be used with the navigation system 10. Alterative tracking systems may include radar tracking systems, acoustic tracking systems, ultrasound tracking systems, and the like. Each of the different tracking systems may include respective different tracking devices and localizers operable with the respective tracking modalities. Also, the different tracking modalities may be used simultaneously as long as they do not interfere with each other (e.g., an opaque member blocks a camera view of optical localizer 52).

With an EM tracking system, EM localizer 50 and the various tracking devices may communicate through an EM controller. The EM controller may include various amplifiers, filters, electrical isolation, and other systems. The EM controller may also control EM coils of EM localizer 50 to either emit or receive an EM field for tracking. The EM controller may be incorporated into a navigation processing system 70. Other potential tracking systems that may be used in navigation system 10 may include an acoustic tracking system, a radiation tracking system, a radar tracking system, an impedance tracking system, or the like.

Navigation system 10 may include a navigation processing unit 74 that may communicate with or include a navigation memory 76, which may be included in navigation processing system 70. Navigation processing system 70 may further include a display device 77. Navigation processing unit 74 may include processing circuitry (e.g., one or more microprocessors, central processing units, etc.). In some examples, navigation processing unit 74 may execute instructions to perform the techniques discussed herein. For example, navigation processing unit 74 may execute instructions to determine at least one property of patient tissue at a location relative to a reference position, localize, using the reference position, the patient tissue relative to at least one of a medical instrument or a medical device, and output, for display a representation of at least one of the medical instrument or the medical device and a representation of the patient tissue, the representation of the patient tissue including a representation of the at least one property.

Navigation processing unit 74 may receive information, including image data, from imaging system 12 and tracking information from the tracking systems, including the respective tracking devices and/or the localizers 50, 52, and/or 55. Image data may be displayed as an image 78 on display device 26. In some examples, image 78 may include colorized representations of patient tissue where the color of the representation of the patient tissue is indicative of at least one property of the patient tissue. In some examples, the at least one property may include at least one of a stiffness or electrical conductivity of the patient tissue. Display device 26 may be separate from and/or integrated into navigation system 10. Navigation processing system 70 may include appropriate input devices, such as a keyboard 84. It will be understood that other appropriate input devices may be included, such as a mouse, a foot pedal 88 or the like which may be used separately or in any combination. Also, all of the disclosed processing units or systems may be a single processor module (e.g., a single central processing chip) or a plurality of processor modules (e.g., more than one central processing chip) that may execute different instructions to perform different tasks.

An image processing unit or module may process image data from imaging system 12. In some examples, an image processor (not shown in FIG. 1) may be provided to process or pre-process image data from imaging system 12. For example, the image processor may colorize patient tissue of the image data to indicate at least one property associated with the patient tissue. The image data from the image processor may be transmitted to navigation processing unit 74. It should be understood, however, that imaging system 12 need not perform any image processing and the image data may be transmitted directly to navigation processing unit 74, which may colorize the patient tissue of the image data. Accordingly, navigation system 10 may include or operate with a single or multiple processing centers or units that may access single or multiple memory systems based upon system design.

In various examples, imaging system 12 may generate image data that may be used to compose image 78 and define an image space that may be registered to a patient space or navigation space that is defined by and/or relative to patient 14. In various examples, the position of patient 14 relative to imaging system 12 may be determined by the navigation system 10 with the patient tracking device 56 and the imaging system tracking device(s) 22 to assist in and/or maintain registration. Accordingly, the position of patient 14 relative to imaging system 12 may be determined.

Manual or automatic registration of the image space to the subject space may occur. In various examples, the registration may occur by matching fiducial points in image data with fiducial points on patient 14. Registration of image space to patient space allows for the generation of a translation map between the patient space and the image space. According to various examples, registration may occur by determining points that are substantially identical in the image space and the patient space. The identical points may include anatomical fiducial points or implanted fiducial points. Exemplary registration techniques are disclosed in U.S. Pat. No. 9,737,235, issued Aug. 22, 2017, which is incorporated herein by reference.

In some examples, imaging system 12 may be used with an un-navigated or navigated procedure. In a navigated procedure, a localizer and/or digitizer, including either or both of an optical localizer 52 and/or an electromagnetic localizer 50, 55 may be used to generate a field and/or receive and/or send a signal within a navigation domain relative to patient 14. The navigated space or navigational domain relative to patient 14 may be registered to image 78. Correlation, as understood in the art, is to allow registration of a navigation space defined within the navigational domain and an image space defined by image 78. Patient tracker 56 may be connected to patient 14 to allow for a dynamic registration and maintenance of registration of patient 14 to image 78 even when patient 14 may move.

Once registered, navigation system 10 with or including imaging system 12, may be used to perform selected procedures, such implantation of an IMD or lead. Selected procedures may use the image data generated or acquired with imaging system 12. Further, imaging system 12 may be used to acquire image data at different times relative to a procedure. As discussed herein, image data may be acquired of patient 14 prior to the procedure for collection of automatically registered image data or cine loop image data. Also, imaging system 12 may be used to acquire images for confirmation of a portion of the procedure. Thus, image data may be acquired at any appropriate time and may be registered to patient 14.

Upon registration and tracking of instrument 62, a graphic representation 90 (e.g., an icon, indicium, animation or other or visual representation) may be displayed relative to, including overlaid (e.g., superimposed) on, image 78. Image 78 may be an appropriate image and may include one or more 2D images, such as 2D images that are acquired at different planes. Images may also be a 3D image, or any appropriate image as discussed herein.

In addition to registering the subject space to the image space, however, an imaging plane of imaging system 12 may also be determined. By registering the image plane of imaging system 12, imaged portions may be located within patient 14. For example, when the image plane is calibrated to tracking device(s) 22 associated with US housing 16 then a position of an imaged portion of heart 15, or other imaged portion, may also be tracked.

Once patient 14 is in condition for a procedure, patient 14 may be positioned on table 40, as noted above. Display device 26 and/or display 77 may display various information regarding patient 14 and/or other information selected by user 18. As noted above, display device 26 may illustrate image 78 that may be acquired with imaging system 12. Image 78 may also be displayed on display 77. Also as noted above, instrument 62 may be tracked and graphic representation 90 of instrument 62 may be displayed on display device 26, such as relative to image 78. In addition, or alternatively, graphic representation 90 may also be displayed relative to a patient avatar 92. Patient avatar 92 may be based on a general avatar that includes various features of patient 14, as noted herein, but is sized to the current and specific patient. Patient avatar 92 may be illustrated as a two-dimensional (2D) image and/or a three-dimensional (3D) image.

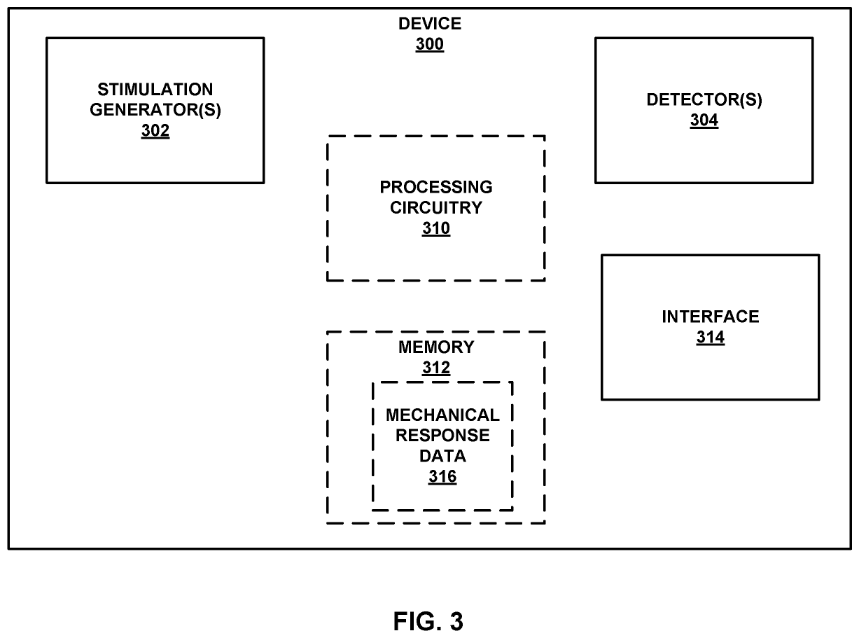

Navigation system 10 may use elastography and/or thermoacoustic imaging (TAI) which, in some examples, may be used alone or together with imaging techniques, such as ultrasound imaging techniques. Both elastography and TAI techniques are sensitive to soft tissue properties, but employ different techniques to sense the soft tissue properties. For example, elastography may include stimulating target tissue with mechanical waves and monitoring a mechanical response of the tissue. This mechanical stimulation may be either induced externally or internally to patient 14, such as through device 300 of FIG. 3, or be induced from motion within the body (e.g., heart valve moving) of patient 14. TAI may include stimulating the target tissue with electromagnetic energy in the microwave or radiofrequency range and monitoring for a mechanical response of the tissue.

For example, navigation system 10 may track an ultrasound imaging probe (e.g., within US housing 16) via navigation techniques (e.g., electromagnetic, electrical impedance, optical, etc.), thereby facilitating localization of the patient tissue. A patient reference tracker (e.g., reference tracking device 56 or reference tracking device 60) may be employed to ensure the tissue remains localized in the case of patient movement. If additional and/or separate devices (e.g., transducers, mechanical stimulation generators, microwave emitters, RF emitters, mechanical wave detectors, etc.) are utilized to perform the elastography or TAI measurements, such devices may be localized with a tracking technology as well.

By tracking the medical instruments, navigation system 10 may localize determined tissue properties and register such properties to the patient anatomy, for example, by ultrasound or other techniques. Navigation system 10 may localize IMDs and/or delivery systems therefor with such tracking techniques and facilitate the navigation of such IMDs relative to these localized tissue properties. For example, navigation system 10 may guide a clinician in positioning a lead delivery catheter or a delivery system for an IMD such as a leadless pacemaker (e.g., a ventricle-from-atrium leadless pacemaker) to avoid lead deployment into less desirable tissue, such as a scarred myocardium.

Navigation system 10 may assign navigation coordinates with respect to a reference position (e.g., a reference coordinate), such as one based on a location of a reference tracking device, such as reference tracking device 56. For example, navigation system 10 may determine a reference coordinate based on the location of reference tracking device 56. For example, navigation system 10 may determine a reference position using a Cartesian coordinate system, such as a reference coordinate 0,0,10. Navigation system 10 may identify fatty tissue at 0,0,0 coordinates (in a Cartesian coordinate system) and identify that fatty tissue on display device 26 so that a clinician may avoid implanting the IMD at that location (e.g., 0,0,0 coordinates). It should be noted that other position systems or coordinate systems may be utilized.

In some examples, imaging system 12 (e.g., US housing 16) or instrument tracking device 60 may include an ultrasound probe and an elastography and/or TAI device. For example, US housing 16 or instrument tracking device 60 may include both an ultrasound imaging device and an elastography and/or TAI device. In such a case, US housing 16 and/or instrument tracking device 60 may include an RF emitter, a microwave emitter, and or a device configured to create mechanical waves in patient 14. In some examples, US housing 16 and/or instrument tracking device 60 may include a device configured to sense a mechanical response from tissue of patient 14 to the RF emissions, microwave emissions, and/or responses to mechanical waves created by one or more stimulation generators of US housing 16 and/or instrument tracking device 60 and/or mechanical motion of patient 14 (such as motion of a heart valve). In some examples, US housing 16 and/or instrument tracking device 60 may utilize the ultrasound probe for ultrasound imaging and for ultrasound elastography. In some examples, the elastography device and/or the TAI device may be part of an IMD, a delivery catheter, or a delivery system for an IMD such as a leadless pacemaker, placed on a body of patient 14 (e.g., external to patient 14), or placed within the body of patient 14 (e.g., internal to patient 14) separate from the IMD and delivery catheter.

In some examples, instrument tracking device 60 may include a tracking element, such as an electromagnetic coil, in a handle for example, to allow tracking in space. Navigation system 10 may assign coordinates to track instrument tracking device 60. Imaging system 12 may display on a display device, such as display device 26, depth and dimension information. For example, navigation system 10 may track a distal tip or other portion of a delivery system or delivery catheter.

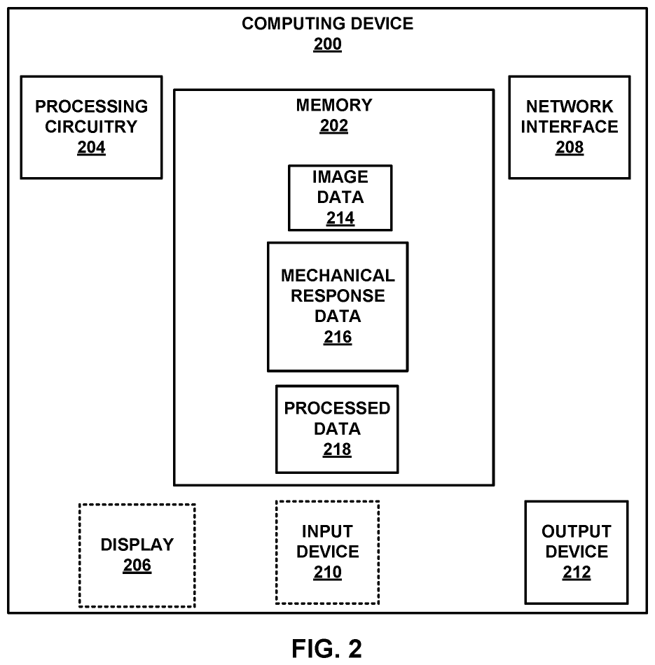

FIG. 2 is a schematic view of an example of navigation processing system according to one or more aspects of this disclosure. Computing device 200 may represent an example of navigation processing system 70 of FIG. 1. Computing device 200 may include a workstation, a desktop computer, a laptop computer, a smart phone, a tablet, a dedicated computing device, or any other computing device capable of performing one or more techniques of this disclosure.

Computing device 200 may include, for example, a memory 202, processing circuitry 204, a display 206, a network interface 208, an input device 210, or an output device 212, for ease of description. While processing circuitry 204 appears in computing device 200 in FIG. 2, in some examples, features attributed to processing circuitry 204 may be performed by processing circuitry of any other device or combination of devices described herein. Additionally, in some examples, processing operations or other operations performed by processing circuitry 204 may be performed by one or more processors residing remotely, such as one or more cloud servers or processors, each of which may be considered a part of computing device 200. Computing device 200 may be used to perform any of the techniques described in this disclosure, and may form all or part of devices or systems configured to perform such methods, alone or in conjunction with other components, such as other components of navigation system 10.

Memory 202 of computing device 200 includes any non-transitory computer-readable storage media for storing data or software that is executable by processing circuitry 204 and that controls the operation of computing device 200, as applicable. In one or more examples, memory 202 may include one or more solid-state storage devices such as flash memory chips. In one or more examples, memory 202 may include one or more mass storage devices connected to the processing circuitry 204 through a mass storage controller (not shown) and a communications bus (not shown).

Although the description of computer-readable media herein refers to a solid-state storage, it should be appreciated by those skilled in the art that computer-readable storage media may be any available media that may be accessed by the processing circuitry 204. That is, computer readable storage media includes non-transitory, volatile and non-volatile, removable and non-removable media implemented in any method or technology for storage of information such as computer-read able instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by computing device 200. In one or more examples, computer-readable storage media may be stored in the cloud or remote storage and accessed using any suitable technique or techniques through at least one of a wired or wireless connection.

Memory 202 may store image data 214. Image data 214 may be captured by an imaging system, such as an imaging system including US housing 16, prior to, during, or after a medical procedure of a patient. Processing circuitry 204 may receive image data 214 from the imaging system and store image data 214 in memory 202. Processing circuitry 204 may receive mechanical response information from one or more detectors (not shown in FIG. 2) and this mechanical response information in mechanical response data 216. Mechanical response data 216 may be indicative of a mechanical response of patient tissue to stimulation. For example, mechanical response data 216 may include a quantification of a magnitude of a mechanical response of a particular portion of patient tissue to which one or more detectors is monitoring. This quantification of a magnitude of a mechanical response may be indicative of one or more properties of the patient tissue, such as a stiffness, level of scarring, fatty composition, electrical conductivity, or the like, of the patient tissue. Processing circuitry 204 may also process image data 214 to colorize or otherwise provide an indication of the at least one property of patient tissue and store the processed image data in processed data 218.

Processing circuitry 204 may be implemented by one or more processors, which may include any number of fixed-function circuits, programmable circuits, or a combination thereof. In various examples, control of any function by processing circuitry 204 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 204 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Display 206 may be an example of display device 77 of FIG. 1. Display 206 may be a touch sensitive or voice activated, enabling display 206 to serve as both an input and output device. Alternatively, a keyboard (not shown in FIG. 2), mouse (not shown in FIG. 2), or other data input devices (e.g., input device 210) may be employed.

Network interface 208 may be adapted to connect to a network such as a local area network (LAN) that includes a wired network or a wireless network, a wide area network (WAN), a wireless mobile network, a Bluetooth network, or the internet. For example, computing device 200 may communicate with other devices of navigation system 10, e.g., receive image data 214 and receive mechanical response data 216 from other devices of navigation system 10 via network interface 208.

Input device 210 may be any device that enables a user to interact with computing device 200, such as, for example, a mouse, keyboard, foot pedal, touch screen, augmented-reality input device receiving inputs such as hand gestures or body movements, or voice interface.

Output device 212 may include any connectivity port or bus, such as, for example, parallel ports, serial ports, universal serial busses (USB), or any other similar connectivity port known to those skilled in the art.

FIG. 3 is a block diagram illustrating an example mechanical response detection device according to one or more aspects of this disclosure. Device 300 may be an example of an elastography and/or thermoacoustic imaging device. In some examples, device 300 may be part of imaging system 12, such as housed within US housing 16. In some examples US housing 16 may not house an ultrasound imager and may instead house or be a housing of device 300. In some examples, device 300 may be configured to remain outside of patient 14. In other examples, device 300 may be configured to be at least partially inserted into patient 14, for example, device 300 may be resident on a medical instrument, IMD, delivery system (e.g., for an IMD such as a leadless pacemaker) or imaging device.

Device 300 may include one or more stimulation generator(s) 302. For example, one or more stimulation generator(s) 302 may include a mechanical stimulation generator and/or an electromagnetic stimulation generator. In the case of a mechanical stimulation generator, one or more stimulation generator(s) 302 may be configured to generate mechanical stimulation through vibration or movement of mechanical components of the mechanical stimulation generator. In the case of an electromagnetic stimulation generator, one or more stimulation generator(s) 302 may be configured to generate electromagnetic stimulation in the form of electromagnetic energy in a microwave frequency range and/or a radio frequency range. Device 300 may utilize stimulation generator(s) 302 to apply stimulation to patient tissue of patient 14.

Device 300 may include one or more detector(s) 304. One or more detector(s) 304 may be configured to sense a mechanical response of the patent tissue to stimulation. In some examples, one or more detector(s) 304 may sense a mechanical response of the patient tissue to stimulation generated by one or more stimulation generator(s) 302. In some examples, one or more detector(s) 304 may sense a mechanical response of the patient tissue to stimulation occurring within patient 14, such as mechanical stimulation from the operation of a heart valve.

In some examples, device 300 may include processing circuitry 310, memory 312, and interface 314. In some examples, processing circuitry 310 may perform some of the functions attributed to processing circuitry 204.

Memory 312 includes any non-transitory computer-readable storage media for storing data or software that is executable by processing circuitry 310 and that controls the operation of device 300, as applicable. In one or more examples, memory 312 may include one or more solid-state storage devices such as flash memory chips.

Although the description of computer-readable media herein refers to a solid-state storage, it should be appreciated by those skilled in the art that computer-readable storage media may be any available media that may be accessed by the processing circuitry 310. That is, computer readable storage media includes non-transitory, volatile and non-volatile, removable and non-removable media implemented in any method or technology for storage of information such as computer-readable instructions, data structures, program modules, or other data. For example, computer-readable storage media includes RAM, ROM, EPROM, EEPROM, flash memory or other solid state memory technology, CD-ROM, DVD, Blu-Ray or other optical storage, magnetic cassettes, magnetic tape, magnetic disk storage or other magnetic storage devices, or any other medium that may be used to store the desired information and that may be accessed by device 300. In one or more examples, computer-readable storage media may be stored in the cloud or remote storage and accessed using any suitable technique or techniques through at least one of a wired or wireless connection, for example, of interface 314.

Memory 312 may store one or more programs which may, under the execution of processing circuitry 310, control one or more stimulation generator(s) to generate stimulation. Memory 312 may store one or more programs which may, under the execution of processing circuitry 310, control one or more detector(s) 304 to sense a mechanical response of the patient tissue. In some examples, memory 312 may store mechanical response data 316. For example, processing circuitry 310 may control stimulation generator(s) 302 to generate stimulation which may be directed to or applied to patient tissue. Detector(s) 304 may detect a mechanical response from the patient tissue to stimulation from stimulation generator(s) 302 and/or to mechanical movement within patient 14. Processing circuitry 310 may receive mechanical response information from detector(s) 304, such as a magnitude of a motion wave, from patient tissue at a location. In some examples, processing circuitry 310 may process the magnitude from the patient tissue at the location prior to storing the processed amplitude in mechanical response data 316. In some examples, processing circuitry 310 may not process the magnitude and may store the raw amplitude into mechanical response data 316. Mechanical response data 316 may therefore be indicative of a mechanical response of patient tissue to stimulation. For example, mechanical response data 316 may include a quantification of a magnitude of a mechanical response of a particular portion of patient tissue to which one or more detectors is monitoring. In some examples, the quantification of the magnitude of the mechanical response of mechanical response data 316 may be on a decibel scale. This quantification of a magnitude of a mechanical response may be indicative of one or more properties of the patient tissue, such as a stiffness, level of scarring, fatty composition, electrical conductivity, or the like, of the patient tissue.

Processing circuitry 310 may be implemented by one or more processors, which may include any number of fixed-function circuits, programmable circuits, or a combination thereof. In various examples, control of any function by processing circuitry 310 may be implemented directly or in conjunction with any suitable electronic circuitry appropriate for the specified function. Fixed-function circuits refer to circuits that provide particular functionality and are preset on the operations that may be performed. Programmable circuits refer to circuits that may programmed to perform various tasks and provide flexible functionality in the operations that may be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Instructions may be executed by one or more processors, such as one or more digital signal processors (DSPs), general purpose microprocessors, application specific integrated circuits (ASICs), field programmable gate arrays (FPGAs), graphics processing units (GPUs) or other equivalent integrated or discrete logic circuitry. Accordingly, the term processing circuitry 310 as used herein may refer to one or more processors having any of the foregoing processor or processing structure or any other structure suitable for implementation of the techniques described herein. In addition, in some aspects, the functionality described herein may be provided within dedicated hardware or software modules configured for encoding and decoding, or incorporated in a combined codec. Also, the techniques could be fully implemented in one or more circuits or logic elements.

Interface 314 may be a network interface similar to network interface 208 of FIG. 2 or may be some other interface for inputting data or control information (e.g., in the example where device 300 does not include processing circuitry 310) into device 300 or outputting data from device 300. For example, interface 314 may include mechanical connectors for connecting wires or optical cables to device 300.

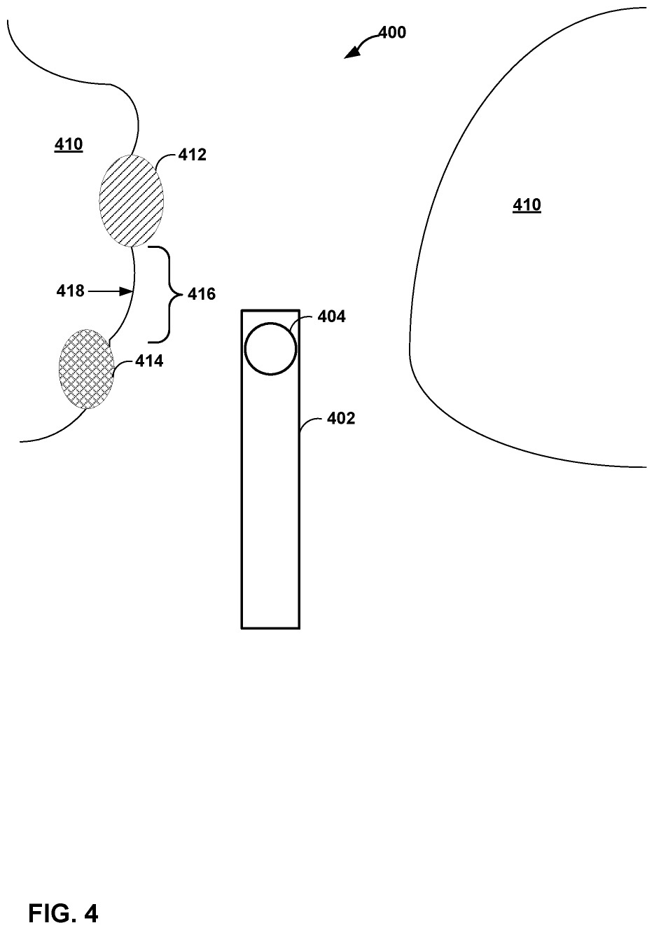

FIG. 4 is a conceptual diagram illustrating a display of a medical instrument, IMD, and patient tissue including a representation of at least one property of the patient tissue according to one or more aspects of this disclosure. Representations 400 may be an example of what may be displayed on a display device such as display device 26. It should be noted that the scale of various items in FIG. 4 may not be consistent between items and may be shown as such for explanatory purposes.

Representations 400 may include a medical instrument representation 402, which may represent a medical instrument such as a delivery catheter, and an IMD representation 404, such as a representation of a ventricle-from-atrium therapy device (e.g. pacemaker), an extravascular implantable cardiac defibrillator, a lead less pacemaker, a lead, or other IMD. Representations 400 may also include a patient tissue representation 410.

In the example of FIG. 4, patient tissue representation 412 may represent patient tissue that is relatively fatty compared to other tissue of patient 14 (which may be represented by patient tissue representation 410). Patient tissue representation 414 may represent patient tissue that is relatively scarred compared to other tissue of patient 14. Processing circuitry 204 may output for display representations 400 such that patient tissue representation 412 appears in a different manner than, for example, an area 416 of patient tissue representation 410 that may represent normal, healthy tissue. Processing circuitry 204 may also output for display representations 400 such that patient tissue representation 414 appears in a different manner than, for example, area 416. In some examples, processing circuitry 204 may output for display representations 400 in a manner that patient tissue representation 412 appears differently than patient tissue representation 414 (e.g., via diagonal fill versus cross-hatched fill as shown). In some examples, rather than indicate patient tissue representation 410 (representing relatively healthy patient tissue with no fill), patient tissue representation 412 (representing relatively fatty patient tissue with diagonal fill), and patient tissue representation 414 (representing relatively scarred patient tissue with cross-hatched fill), processing circuitry 204 may colorize at least a portion of the patient tissue representations to indicate the at least one property (e.g., stiffness) of the displayed tissue. For example, processing circuitry 204 may indicate the actual patient tissue corresponding to patient tissue representation 412 is fatty by coloring patient tissue representation 412 a particular color, such as red. Processing circuitry 204 may also indicate the actual patient tissue corresponding to patient tissue representation 414 is scarred by coloring patient tissue representation 414 a different color, such as blue, or a different shade of the same color as patient tissue representation 412.

In some examples, processing circuitry 204 may colorize patient tissue representation 412 and patient tissue representation 414 the same color, such as red, to indicate this represented tissue that is not very electrically conductive (which may be a property of the tissue) and should be avoided as an implantation site. In some examples, processing circuitry 204 may colorize patient tissue representation 410 as well, such as green, which may indicate that the actual patient tissue represented by patient tissue representation 410 (e.g., area 416) is generally more electrically conductive than other areas, such as the actual tissue represented by patient tissue representation 412 and patient tissue representation 414.

In some examples, processing circuitry 204 may colorize representations of patient tissue along a continuum of colors and/or shades to represent various degrees of mechanical response to stimulus sensed by one or more detector(s) 304. For example, rather than output representations 400 for display in two or three colors, processing circuitry 204 may output representations 400 for display in any number of colors, shades, or the like. For example, actual patient tissue corresponding to point 418 may be more electrically conductive than other actual patient tissue corresponding to points in area 416 as point 418 may be further from fatty patient tissue (e.g., represented by patient tissue representation 412) and scarred patient tissue (e.g., represented by patient tissue representation 414). Processing circuity 204 may therefore indicate point 418 with a brighter, darker or different color than other points in area 416 so as to help guide a clinician to deliver an IMD to the location of patient tissue represented by point 418.

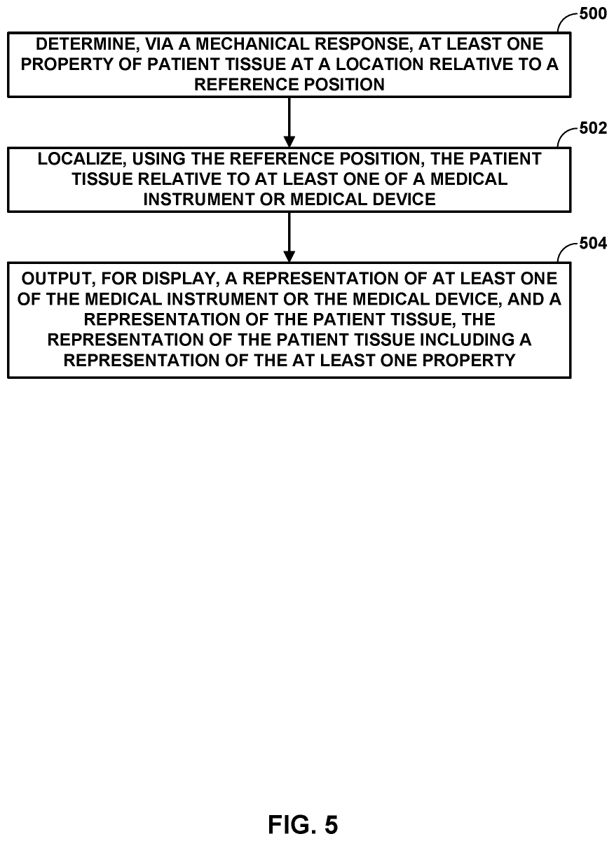

FIG. 5 is a flow diagram illustrating example tissue property identification techniques in accordance with one or more aspects of this disclosure. Processing circuitry 204 may determine, via a mechanical response, at least one property of patient tissue at a location relative to a reference position (500). For example, processing circuitry 204 may determine a reference position, such as a reference coordinate, using any of EM localizer 50, optical localizer 52, EM localizer 55, reference tracking device 56, and/or reference tracking device 60 and a location of the patient tissue relative to the reference position (e.g., reference coordinate). Processing circuitry 204 may monitor a mechanical response of the patient tissue to stimulation via one or more detector(s) 304. Processing circuitry 204 may utilize the mechanical response of the patient tissue to the stimulation to determine the at least one property of the patient tissue. For example, processing circuitry 204 may determine that the patient tissue is fatty if the mechanical response to the stimulation is relatively high. Additionally, processing circuitry 204 may determine the patient tissue is scarred if the mechanical response to the stimulation is relatively low.

Processing circuitry 204 may localize, using the reference position, the patient tissue relative to at least one of a medical instrument or a medical device (502). For example, processing circuitry 204 may determine the location of the patient tissue and at least one of the medical instrument of the medical device relative to the reference position.

Processing circuitry 204 may output, for display, a representation of at least one of the medical instrument or the medical device, and a representation of the patient tissue, the representation of the patient tissue comprising a representation of the at least one property (504). For example, processing circuitry 204 may output a representation of a delivery catheter, an IMD, a lead, and/or the like and a representation of the patient tissue. The representation of the patient tissue may include information, such as color, which may indicate the at least one property, such as stiffness.

In some examples, as part of determining the at least one property of the patient tissue, processing circuitry 204 is configured to monitor a mechanical response of the patient tissue to stimulation. In some examples, as part of determining the at least one property of the patient tissue, processing circuitry 204 is further configured to compare the mechanical response to at least one predetermined threshold and to determine the at least one property of the patient tissue based on the comparison. In some examples, the at least one predetermined threshold comprises a first stiffness threshold and a second stiffness threshold. For example, the first stiffness threshold may be a relatively higher threshold and a mechanical response that meets the first stiffness threshold (e.g., is greater than or greater than or equal to) may be indicative that the patient tissue is less stiff than healthy tissue and/or is fatty tissue. The second stiffness threshold may be a relatively lower threshold and a mechanical response that meets the second stiffness threshold (e.g., is less than or less than or equal to) may be indicative that the patient tissue is more stiff than healthy tissue and/or is scar tissue.

In some examples, the stimulation includes at least one of mechanical stimulation or electromagnetic stimulation. For example, the stimulation may be mechanical stimulation generated by patient 14 itself (e.g., a heart valve of patient 14) or by an elastography device, or may be electromagnetic stimulation generated by a thermoacoustic imaging device. In some examples, processing circuitry 204 is further configured to control a stimulation generator (e.g., of one or more stimulation generator(s) 302) to deliver the stimulation to the patient tissue. In some examples, the electromagnetic stimulation includes electromagnetic energy in at least one of a microwave frequency range or a radio frequency range.

In some examples, as part of determining the properties of the patient tissue, processing circuitry 204 may utilize at least one of elastography and thermoacoustic imaging. In some examples, as part of outputting for display the representation of the patient tissue, processing circuitry 204 may determine a colorized representation of the patient tissue, based on imaging data and the determined at least one property of the patient tissue and output, for display, the colorized representation of the patient tissue.

In some examples, processing circuitry 204 may track motion of the at least one of the medical instrument or medical device and output for display an updated representation of the at least one of the medical instrument or the medical device based on the motion of the at least one of the medical instrument or the medical device.

In some examples, the reference position includes a reference coordinate. In some examples, as part of localizing the patient tissue relative to at least one of the medical instrument or the medical device, processing circuitry 204 may localize the patient tissue relative to at least one of a plane of the medical instrument or a plan of the medical device. In some examples, as part of localizing the patient tissue relative to at least one of the medical instrument or the medical device, processing circuitry 204 may localize the patient tissue relative to at least one of a direction of travel of the medical instrument or a direction of travel of the medical device.

In some examples, at least one of the medical device includes a leadless pacemaker or the medical instrument includes a delivery system for a leadless pacemaker. In some examples, the medical device includes a ventricle-from-atrium leadless pacemaker. In some examples, the location includes the triangle of Koch. In some examples, the imaging system includes a navigation system or is part of the navigation system.

Example examples are provided so that this disclosure will be thorough and will fully convey the scope to those who are skilled in the art. Numerous specific details are set forth such as examples of specific components, devices, and methods, to provide a thorough understanding of examples of the present disclosure. It will be apparent to those skilled in the art that specific details need not be employed, that example examples may be embodied in many different forms and that neither should be construed to limit the scope of the disclosure. In some example examples, well-known processes, well-known device structures, and well-known technologies are not described in detail.

The foregoing description of the examples has been provided for purposes of illustration and description. It is not intended to be exhaustive or to limit the invention. Individual elements or features of a particular example are generally not limited to that particular example, but, where applicable, are interchangeable and may be used in a selected example, even if not specifically shown or described. The same may also be varied in many ways. Such variations are not to be regarded as a departure from the invention, and all such modifications are intended to be included within the scope of the invention.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit that may also be referred to as a processor. Computer-readable media may include non-transitory computer-readable media, which corresponds to a tangible medium such as data storage media (e.g., RAM, ROM, EEPROM, flash memory, or any other medium that may be used to store desired program code in the form of instructions or data structures and that may be accessed by a computer).

Instructions may be executed by one or more processors or processor modules, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” or “processor module” as used herein may refer to any of the foregoing structure or any other physical structure suitable for implementation of the described techniques. Also, the techniques could be fully implemented in one or more circuits or logic elements.

This disclosure includes the following non-limiting examples.

Example 1. A system comprising: memory configured to store a representation of patient tissue; and processing circuitry communicatively coupled to the memory, the processing circuitry being configured to: determine, via a mechanical response, at least one property of the patient tissue at a location relative to a reference position; localize, using the reference position, the patient tissue relative to at least one of a medical instrument or a medical device; and output, for display, a representation of at least one of the medical instrument or the medical device and a representation of the patient tissue, the representation of the patient tissue comprising a representation of the at least one property.

Example 2. The system of example 1, wherein as part of determining the at least one property of the patient tissue, the processing circuitry is configured to monitor a mechanical response of the patient tissue to stimulation.

Example 3. The system of example 2, wherein as part of determining the at least one property of the patient tissue, the processing circuitry is further configured to: compare the mechanical response to at least one predetermined threshold; and determine the at least one property of the patient tissue based on the comparison.

Example 4. The system of example 3, wherein the at least one predetermined threshold comprises a first stiffness threshold and a second stiffness threshold.

Example 5. The system of any of examples 2-4, wherein the stimulation comprises at least one of mechanical stimulation or electromagnetic stimulation.

Example 6. The system of example 5, wherein the processing circuitry is further configured to control a stimulation generator to deliver the stimulation to the patient tissue.

Example 7. The system of example 5 or example 6, wherein the electromagnetic stimulation comprises electromagnetic energy in at least one of a microwave frequency range or a radio frequency range.

Example 8. The system of any of examples 1-7, wherein as part of determining the at least one property of the patient tissue, the processing circuitry is configured to utilize at least one of elastography and thermoacoustic imaging.

Example 9. The system of any of examples 1-8, wherein as part of outputting for display the representation of the patient tissue, the processing circuitry is configured to: determine a colorized representation of the patient tissue, based on imaging data and the determined at least one property of the patient tissue; and output, for display, the colorized representation of the patient tissue.

Example 10. The system of any of examples 1-9, wherein the processing circuitry is further configured to: track motion of the at least one of the medical instrument or the medical device; and output for display an updated representation of the at least one of the medical instrument or the medical device based on the motion of the at least one of the medical instrument or the medical device.

Example 11. The system of any of examples 1-10, wherein the reference position comprises a reference coordinate.

Example 12. The system of any of examples 1-11, wherein as part of localizing the patient tissue relative to at least one of the medical instrument or the medical device, the processing circuitry is configured to localize the patient tissue relative to at least one of a plane of the medical instrument or a plane of the medical device.

Example 13. The system of any of examples 1-12, wherein as part of localizing the patient tissue relative to at least one of the medical instrument or the medical device, the processing circuitry is configured to localize the patient tissue relative to at least one of a direction of travel of the medical instrument or a direction of travel of the medical device.

Example 14. The system of any of examples 1-13, wherein at least one of the medical device comprises a leadless pacemaker or the medical instrument comprises a delivery system for a leadless pacemaker.

Example 15. The system of example 14, wherein the medical device comprises a ventricle-from-atrium leadless pacemaker.

Example 16. The system of example 14 or example 15, wherein the location comprises the triangle of Koch.

Example 17. A method comprising: determining, via a mechanical response and by an imaging system, at least one property of patient tissue at a location relative to a reference position; localizing, by the imaging system, using the reference position, the patient tissue relative to at least one of a medical instrument or a medical device; and outputting, by the imaging system, for display a representation of at least one of the medical instrument or the medical device and a representation of the patient tissue, the representation of the patient tissue comprising a representation of the at least one property.

Example 18. The method of example 17, wherein determining the at least one property of the patient tissue comprises monitoring a mechanical response of the patient tissue to stimulation.

Example 19. The method of example 18, wherein determining the at least one property of the patient tissue further comprises: comparing, by the imaging system, the mechanical response to at least one predetermined threshold; and determining, by the imaging system, the at least one property of the patient tissue based on the comparison.

Example 20. The method of example 19, wherein the at least one predetermined threshold comprises a first stiffness threshold and a second stiffness threshold.

Example 21. The method of any of examples 18-22, wherein the stimulation comprises at least one of mechanical stimulation or electromagnetic stimulation.

Example 22. The method of example 21, further comprising delivering the stimulation to the patient tissue.

Example 23. The method of example 21 or example 22, wherein the electromagnetic stimulation comprises electromagnetic energy in at least one of a microwave frequency range or a radio frequency range.

Example 24. The method of any of examples 17-23, wherein the determining the at least one property of the patient tissue comprises performing at least one of elastography and thermoacoustic imaging.

Example 25. The method of any of examples 17-24, wherein outputting for display the representation of the patient tissue comprises: determining, by the imaging system, a colorized representation of the patient tissue, based on imaging data and the determined at least one property of the patient tissue; and outputting, for display and by the imaging system, the colorized representation of the patient tissue.

Example 26. The method of any of examples 17-25, further comprising: tracking, by the imaging system, motion of the at least one of the medical instrument or the medical device; and outputting, by the imaging system, for display an updated representation of the at least one of the medical instrument or the medical device based on the motion of the at least one of the medical instrument of the medical device.

Example 27. The method of any of examples 17-26, wherein the imaging system comprises a navigation system or is part of the navigation system.

Example 28. The method of any of examples 17-27, wherein the reference position comprises a reference coordinate.

Example 29. The method of any of examples 17-28, wherein localizing the patient tissue relative to at least one of the medical instrument or the medical device comprises localizing the patient tissue relative to at least one of a plane of the medical instrument or a plane of the medical device.

Example 30. The method of any of examples 17-29, wherein localizing the patient tissue relative to at least one of the medical instrument or the medical device comprises localizing the patient tissue relative to at least one of a direction of travel of the medical instrument or a direction of travel of the medical device.

Example 31. The method of any of examples 17-30, wherein at least one of the medical device comprises a leadless pacemaker or the medical instrument comprises a delivery system for a leadless pacemaker.

Example 32. The method of example 31, wherein the medical device comprises a ventricle-from-atrium leadless pacemaker.

Example 33. The method of example 31 or example 32, wherein the location comprises the triangle of Koch.

Example 34. A non-transitory computer-read able storage medium storing instructions, which, when executed, cause processing circuitry to: determine, via a mechanical response, at least one property of patient tissue relative to a reference coordinate; localize, using the reference coordinate, the patient tissue relative to at least one of a plane of a medical instrument or a medical device; and output, for display, a representation of at least one of the medical instrument or the medical device and a representation of the patient tissue, the representation of the patient tissue comprising a representation of the at least one property.

1. A system comprising:
memory configured to store a representation of patient tissue; and
processing circuitry communicatively coupled to the memory, the processing circuitry being configured to:
determine, via a mechanical response, at least one property of the patient tissue at a location relative to a reference position;
localize, using the reference position, the patient tissue relative to at least one of a medical instrument or a medical device; and
output, for display, a representation of at least one of the medical instrument or the medical device and a representation of the patient tissue, the representation of the patient tissue comprising a representation of the at least one property.

2. The system of claim 1, wherein as part of determining the at least one property of the patient tissue, the processing circuitry is configured to monitor a mechanical response of the patient tissue to stimulation.

3. The system of claim 2, wherein as part of determining the at least one property of the patient tissue, the processing circuitry is further configured to:
compare the mechanical response to at least one predetermined threshold; and
determine the at least one property of the patient tissue based on the comparison.

4. The system of claim 3, wherein the at least one predetermined threshold comprises a first stiffness threshold and a second stiffness threshold.

5. The system of claim 2, wherein the stimulation comprises at least one of mechanical stimulation or electromagnetic stimulation.

6. The system of claim 5, wherein the processing circuitry is further configured to control a stimulation generator to deliver the stimulation to the patient tissue.

7. The system of claim 5, wherein the electromagnetic stimulation comprises electromagnetic energy in at least one of a microwave frequency range or a radio frequency range.

8. The system of claim 1, wherein as part of determining the at least one property of the patient tissue, the processing circuitry is configured to utilize at least one of elastography and thermoacoustic imaging.

9. The system of claim 1, wherein as part of outputting for display the representation of the patient tissue, the processing circuitry is configured to:
determine a colorized representation of the patient tissue, based on imaging data and the determined at least one property of the patient tissue; and
output, for display, the colorized representation of the patient tissue.

10. The system of claim 1, wherein the processing circuitry is further configured to:
track motion of the at least one of the medical instrument or the medical device; and
output for display an updated representation of the at least one of the medical instrument or the medical device based on the motion of the at least one of the medical instrument or the medical device.

11. The system of claim 1, wherein the reference position comprises a reference coordinate.

12. The system of claim 1, wherein as part of localizing the patient tissue relative to at least one of the medical instrument or the medical device, the processing circuitry is configured to localize the patient tissue relative to at least one of a plane of the medical instrument or a plane of the medical device.

13. The system of claim 1, wherein as part of localizing the patient tissue relative to at least one of the medical instrument or the medical device, the processing circuitry is configured to localize the patient tissue relative to at least one of a direction of travel of the medical instrument or a direction of travel of the medical device.

14. The system of claim 1, wherein at least one of the medical device comprises a leadless pacemaker or the medical instrument comprises a delivery system for the leadless pacemaker, and wherein the location comprises a triangle of Koch.

15. The system of claim 14, wherein the medical device comprises a ventricle-from-atrium leadless pacemaker, and wherein the location comprises the triangle of Koch.

16. A method comprising:
determining, via a mechanical response and by an imaging system, at least one property of patient tissue at a location relative to a reference position;
localizing, by the imaging system, using the reference position, the patient tissue relative to at least one of a medical instrument or a medical device; and
outputting, by the imaging system, for display a representation of at least one of the medical instrument or the medical device and a representation of the patient tissue, the representation of the patient tissue comprising a representation of the at least one property.

17. The method of claim 16, wherein determining the at least one property of the patient tissue comprises monitoring a mechanical response of the patient tissue to stimulation.

18. The method of claim 17, wherein determining the at least one property of the patient tissue further comprises:
comparing, by the imaging system, the mechanical response to at least one predetermined threshold; and
determining, by the imaging system, the at least one property of the patient tissue based on the comparison.

19. The method of claim 18, wherein the at least one predetermined threshold comprises a first stiffness threshold and a second stiffness threshold.

20. A non-transitory computer-readable storage medium storing instructions, which, when executed, cause processing circuitry to:
determine, via a mechanical response, at least one property of patient tissue relative to a reference position;
localize, using the reference position, the patient tissue relative to at least one of a medical instrument or a medical device; and
output, for display, a representation of at least one of the medical instrument or the medical device and a representation of the patient tissue, the representation of the patient tissue comprising a representation of the at least one property.
